(19) United States
(12) Patent Application Publication
(10) Pub. No.: US 2012/0189746 A1
(43) Pub. Date: Jul. 26, 2012

(54) STIRRING APPARATUS FOR COOKING VESSEL

(76) Inventor: Timothy DeLong, Boynton Beach, FL (US)

(21) Appl. No.: 13/081,173

(22) Filed: Apr. 6, 2011

Related U.S. Application Data
(63) Continuation-in-part of application No. 13/010,126, filed on Jan. 20, 2011.

(51) Int. Cl. A47J 36/00; B01F 15/00; G01N 33/02; A23P 1/00; A23L 1/01; B01F 7/06; A47J 27/00

(52) U.S. Cl. 426/231; 366/309; 366/270; 366/142; 99/324; 99/348; 99/342; 426/519

(57) ABSTRACT

A stirring assembly, including a vessel and a stirring apparatus. The vessel includes a baffle plate fixed to an interior wall of the vessel. The stirring apparatus, includes: a lid assembly including a variable speed motor, the lid assembly removeably securable to the vessel; a shaft connected to the motor and extending into an interior space of the vessel; and a single scrapper attached to a distal end of the shaft, extending radially outward from the shaft, and in contact with a bottom interior surface of the vessel.

### CROSS-REFERENCE TO RELATED APPLICATIONS

This application is a continuation-in-part of U.S. patent application Ser. No. 13/010,126, filed Jan. 20, 2011, entitled, “STIRRING APPARATUS FOR COOKING VESSEL”, which application is incorporated herein by reference.

### FIELD OF THE INVENTION

The invention broadly relates to a stirring apparatus, and more particularly, to a motorized stirring apparatus in combination with a commercial cooking vessel.

### BACKGROUND OF THE INVENTION

FIG. 1 is a side perspective view of stratified layers generated by prior stirring devices. Stirring apparatus are known in the art. Generally, a stirring apparatus includes a motor, shaft, and blade or propeller, and is used in combination with a cooking vessel. During a cooking process, heavier particulates in the product being cooked settle on the bottom of the cooking vessel, while less dense liquids and particulates rise to the surface. The principal of operation of prior stirring devices and the intended purpose of prior stirring devices is the moving of the settled particulates only around the bottom of the cooking vessel, resulting stratification of the food product, burned food particulates, and an inconsistent end food product.

For example, as shown in FIG. 1, rotation of paddles P in direction R about shaft S creates stratified layers L1 through L5. Adjacent layers are separate and distinct in horizontal direction H, and respective particulate in layers L1 through L5 do not mix. Specifically, the heaviest particulate, found in layer L1, is displaced only in direction R, and not upward to layer L2 and beyond. Thus, particulate in layer L1 remains in contact with the bottom B of vessel V for the duration of the cooking event, resulting in the burning and inconsistent food product noted above. Stated otherwise, the action of paddles P creates a vortex pattern with separate layers L1 through L5 in which particulate is not moved upward in direction H.

The principal of operation of prior stirring devices also involves a single speed for a stirring element. That is, prior stirring devices teach against speed control for a stirring element.

### BRIEF SUMMARY OF THE INVENTION

The present invention broadly comprises a stirring assembly, including a vessel and a stirring apparatus. The vessel includes a baffle plate fixed to an interior wall of the vessel. The stirring apparatus includes: a lid assembly including a variable speed motor, the lid assembly removeably securable to the vessel; a shaft removeably connected to the motor and extending into an interior space of the vessel; and a single scrapper attached to a distal end of the shaft, extending radially outward from the shaft, and in contact with a bottom interior surface of the vessel.

The present invention broadly comprises a cooking vessel, including an interior wall and a baffle plate fixedly connected to the interior wall.

The present invention broadly comprises a method for stirring contents of a cooking vessel, including: positioning a propeller along a length of a shaft; extending the shaft into an interior space of the cooking vessel; contacting a bottom surface of the interior space with a single scrapper, attached to a distal end of the shaft and extending radially outward from the shaft; and rotating, using a variable speed motor, the propeller and the scraper such that: a first portion of the contents located between the propeller and a top of the vessel, in a direction parallel to the shaft, is drawn toward the propeller and displaced past the propeller toward the bottom surface; and a second portion of the contents in contact with the scraper is displaced upward and beyond the propeller in the direction parallel to the shaft.

### BRIEF DESCRIPTION OF THE DRAWINGS

The nature and mode of operation of the present invention will now be more fully described in the following detailed description of the invention taken with the accompanying drawing figures, in which:

FIG. 1 is a side perspective view of stratified layers generated by prior stirring devices;

FIG. 2 is a front exploded view of a cooking vessel and stirring apparatus;

FIG. 3 is a side exploded view of a cooking vessel and stirring apparatus shown in FIG. 2, with a wall of the vessel removed;

FIG. 3A is a detail of area 3A shown in FIG. 3;

FIG. 4 is a front view of the cooking vessel and stirring apparatus shown in FIG. 2, with a wall of the vessel removed;

FIG. 4A is a detail of area 4A shown in FIG. 4;

FIG. 5 is a schematic drawing of the electrical system for the stirring apparatus shown in FIG. 2;

FIG. 6A is a top view of the cooking vessel and stirring apparatus shown in FIG. 2 with the secondary lid removed;

FIG. 6B is a top view of the secondary lid shown in FIG. 2;

FIGS. 7A, 7B, and 7C are front, side, and top views respectively of area 7 in FIG. 2;

FIG. 8 is an exploded cross-section generally of area 8 in FIG. 4; and,

FIG. 9 is a side view of mixing patterns generated by the cooking vessel and stirring apparatus shown in FIG. 2.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and, as such, may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs.

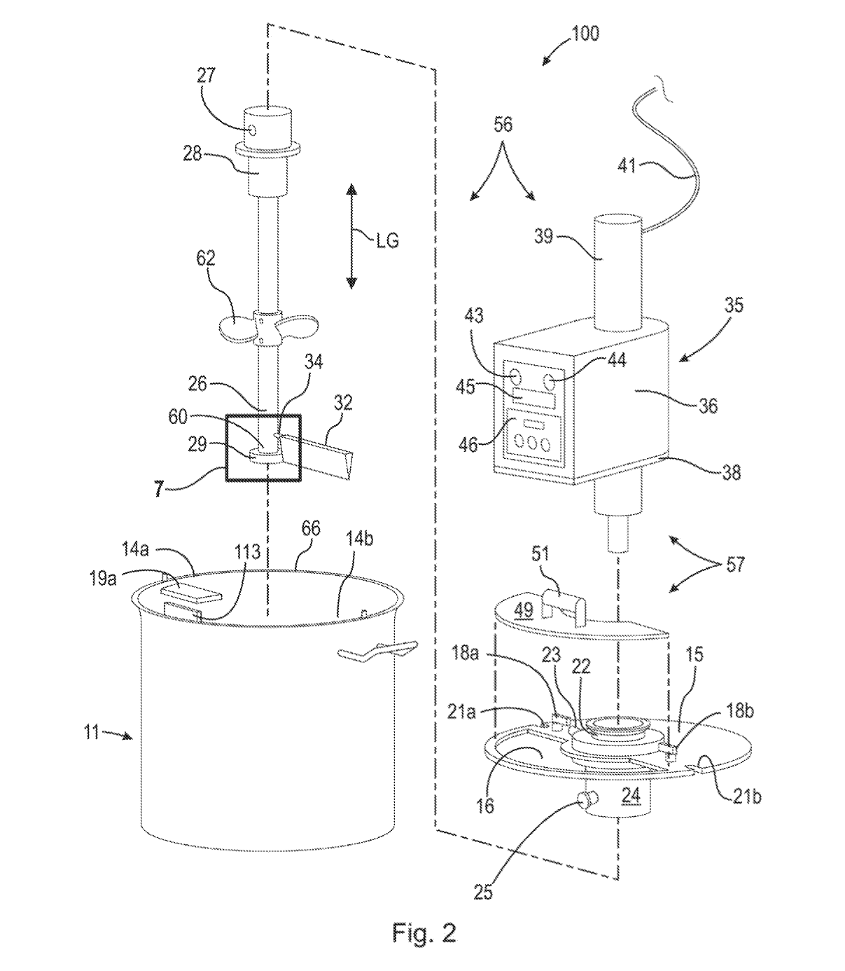

FIG. 2 is a front exploded view of a cooking vessel and stirring apparatus.

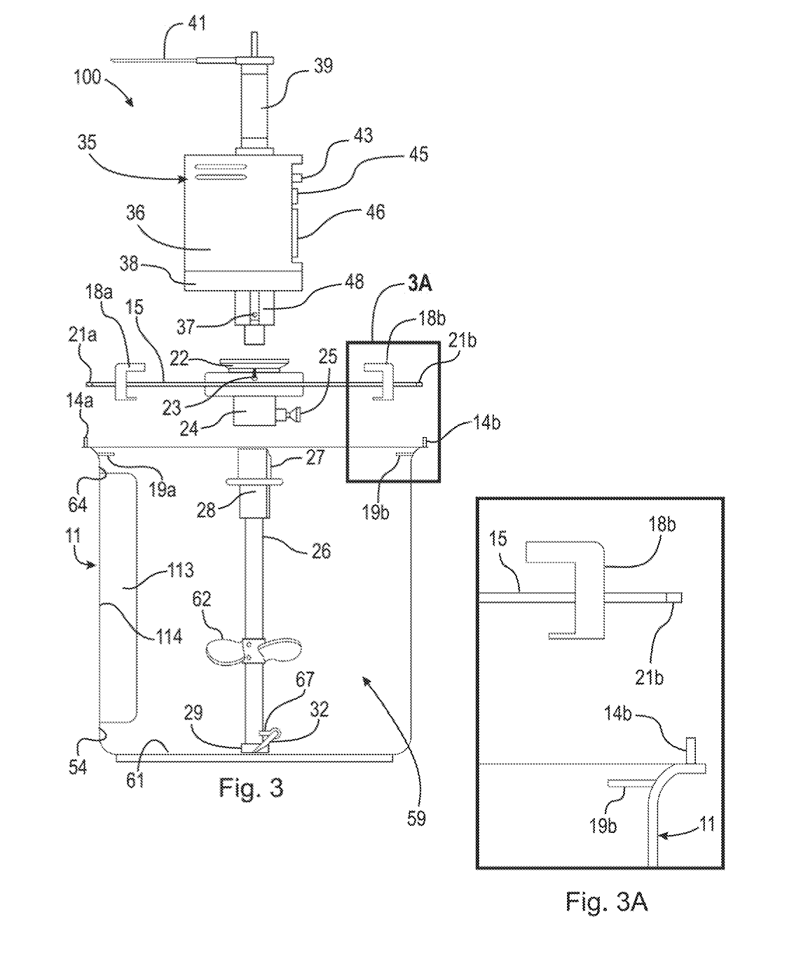

FIG. 3 is a side exploded view of a cooking vessel and stirring apparatus shown in FIG. 2, with a wall of the vessel removed. FIG. 3A is a detail of area 3A shown in FIG. 3.

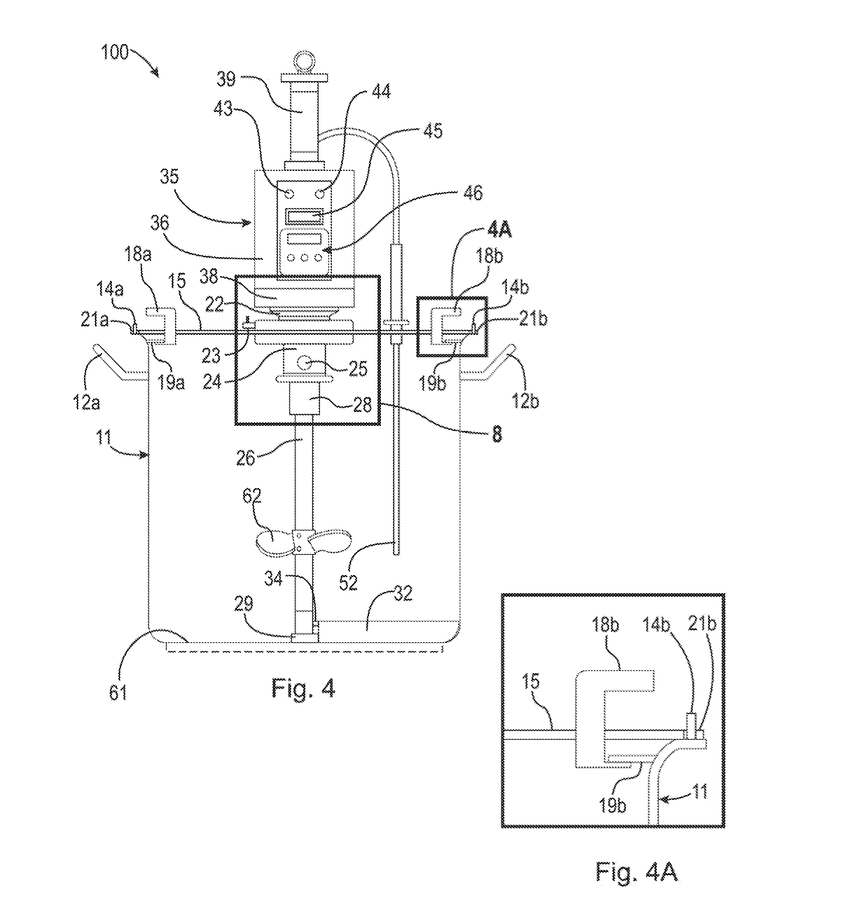

FIG. 4 is a front view of the cooking vessel and stirring apparatus shown in FIG. 2, with a wall of the vessel removed. FIG. 4A is a detail of area 4A shown in FIG. 4. The following should be viewed in light of FIGS. 2 through 4A. Stirring assembly 100 includes cooking vessel 11 with baffle plate 113 fixedly connected to interior wall 54 of the vessel by any means known in the art. In an example embodiment, side 114 of baffle plate 113, facing the interior wall, for example, facing the point of contact with the wall, is in continuous contact with wall 54. Stated otherwise, there is no opening between the baffle and the wall. In an example embodiment, baffle 113 is substantially orthogonal to wall 54. By “substantially orthogonal”, we mean that the baffle is orthogonal to the wall within reasonable fabricating tolerances or substantial portions of the baffle are orthogonal to the wall. It should be understood that other orientations of the baffle with the wall are possible.

Stirring apparatus 56 includes lid assembly 57 removeably securable to the vessel. That is, assembly 57 can be placed on the vessel and secured to the vessel, and assembly 57 can be unsecured from the vessel and removed from the vessel without the use of tools.

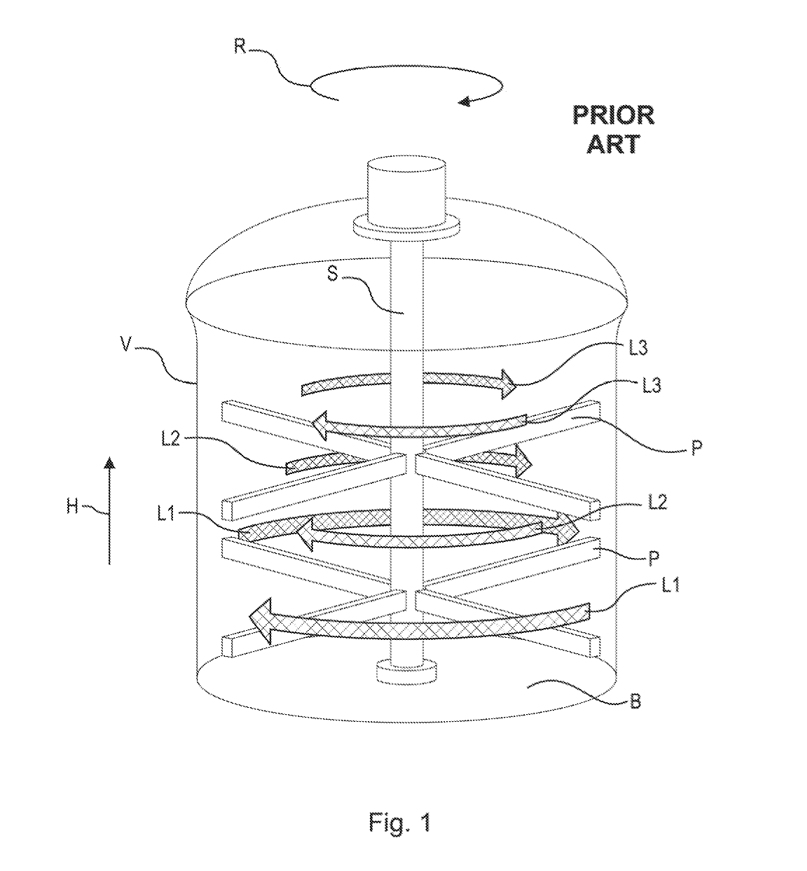

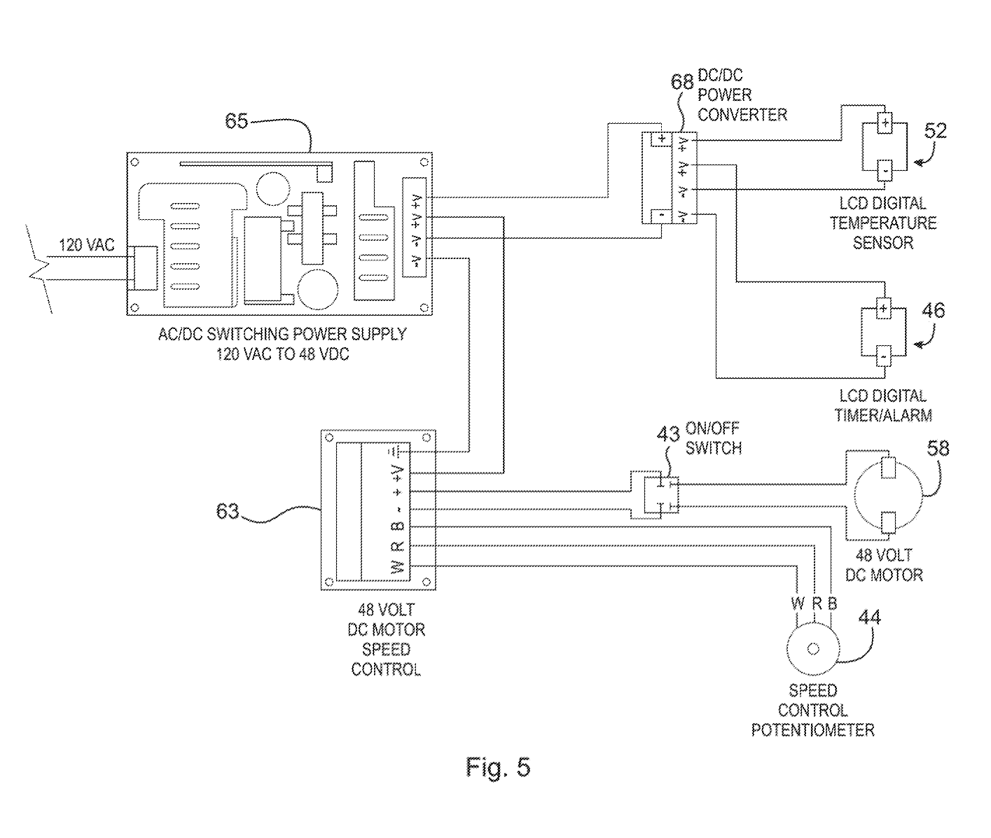

FIG. 5 is a schematic drawing of the electrical system for the stirring apparatus shown in FIG. 1. The following should be viewed in light of FIGS. 2 through 5. The lid assembly includes motor 58 in control unit 35. In an example embodiment, the motor is a variable speed motor. In an example embodiment, the motor is a heavy-duty DC planetary gear motor with a power rating of 200 Watts and a continuous torque rating of 58 inch/pounds. Apparatus 56 also includes shaft 26 connected to the motor and extending into interior space 59 of the vessel. In an example embodiment, the shaft is removeably connected to the motor. That is, the shaft can be attached to and unattached from the motor as further described below. Apparatus 56 includes single scraper 32 attached to distal end 60 of the shaft. The scraper extends radially outward from the shaft and is in contact with bottom interior surface 61 of the vessel. Apparatus 56 includes multi-bladed propeller 62 secured to the shaft and rotatable with the shaft. A position of the propeller along length LG of the shaft is adjustable, for example, through the use of two or more setscrews. In an example embodiment, propeller 62 is a square pitch axial flow propeller.

In an example embodiment, the lid assembly includes thermal sensor 52 extending into interior space 59 of the vessel. In an example embodiment, the control unit includes variable speed control unit 63 for controlling a speed of the motor, temperature display 45 for displaying output from the thermal sensor, that is, the temperature of matter with which the sensor is in contact, for example, food in the vessel. In an example embodiment, the motor operates between zero revolutions per minute (rpm) and 415 rpm. For example, lower speeds can be used for sautéing of food products or stirring of delicate ingredients. Unit 63 can be any speed control unit known in the art. In an example embodiment, unit 63 is an MMP 25A-48 VDC manufactured by Midwest Motion Products.

Unit 35 also includes timer and alarm system 46. In an example embodiment, display 45 displays information regarding time, control parameters, and alarm parameters. For example, a time during which the shaft is to be rotated can be set using system 46. Set points for alarms, such as temperature as sensed by sensor 52, can be set using system 46 and alarms can be expressed, for example, graphically and/or audibly, using system 46. In an example embodiment, converter 68 is used to supply low voltage or low current power for instrumentation, such as sensor 52 and system 46. For example, converter 68 converts 48 VDC power from power supply 65 to 5 VDC.

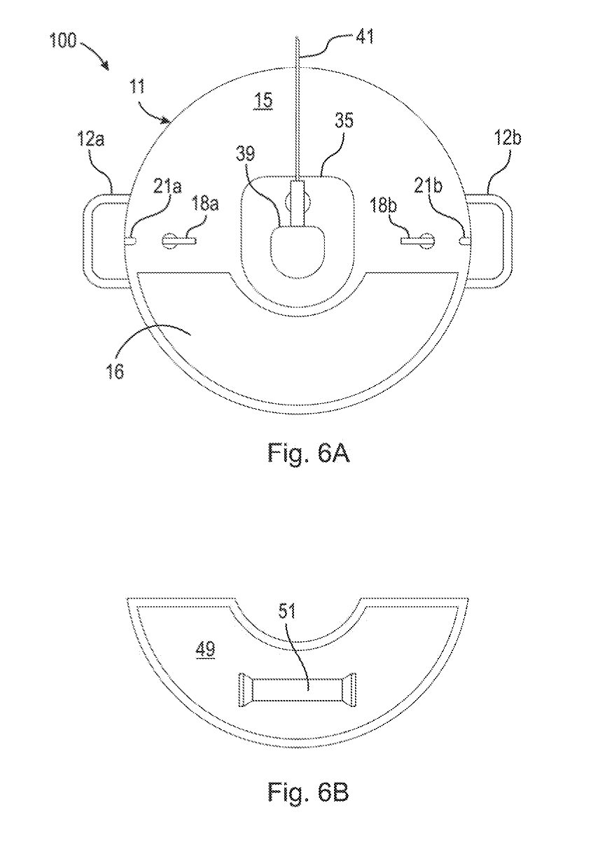

FIG. 6A is a top view of the cooking vessel and stirring apparatus shown in FIG. 2 with the secondary lid removed.

FIG. 6B is a top view of the secondary lid shown in FIG. 2.

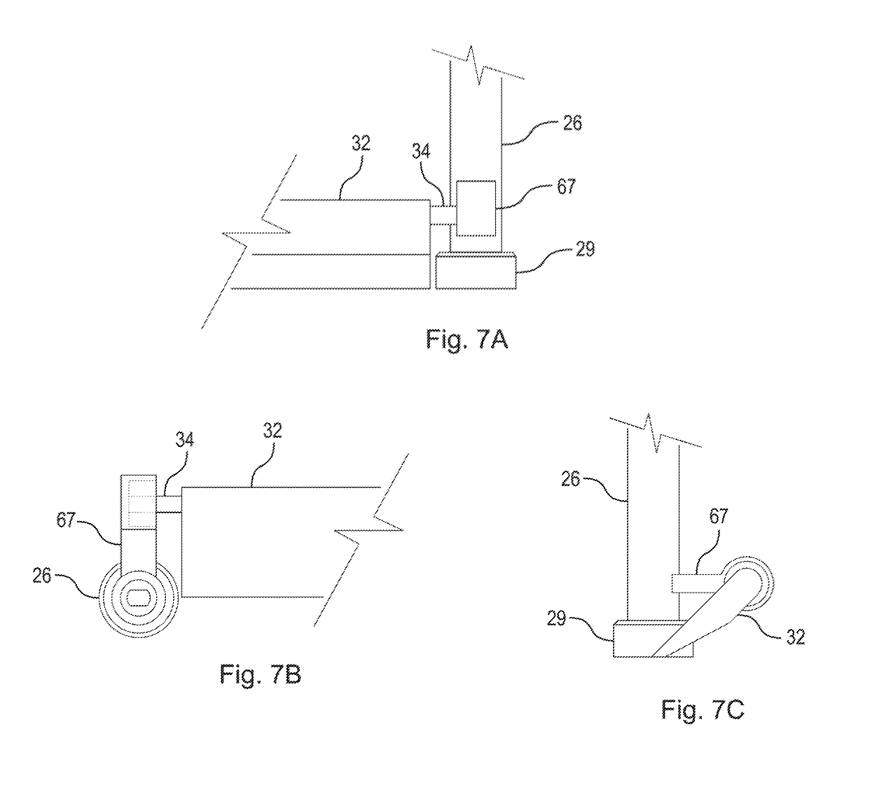

FIGS. 7A, 7B, and 7C are front, side, and top views respectively of area 7 in FIG. 2.

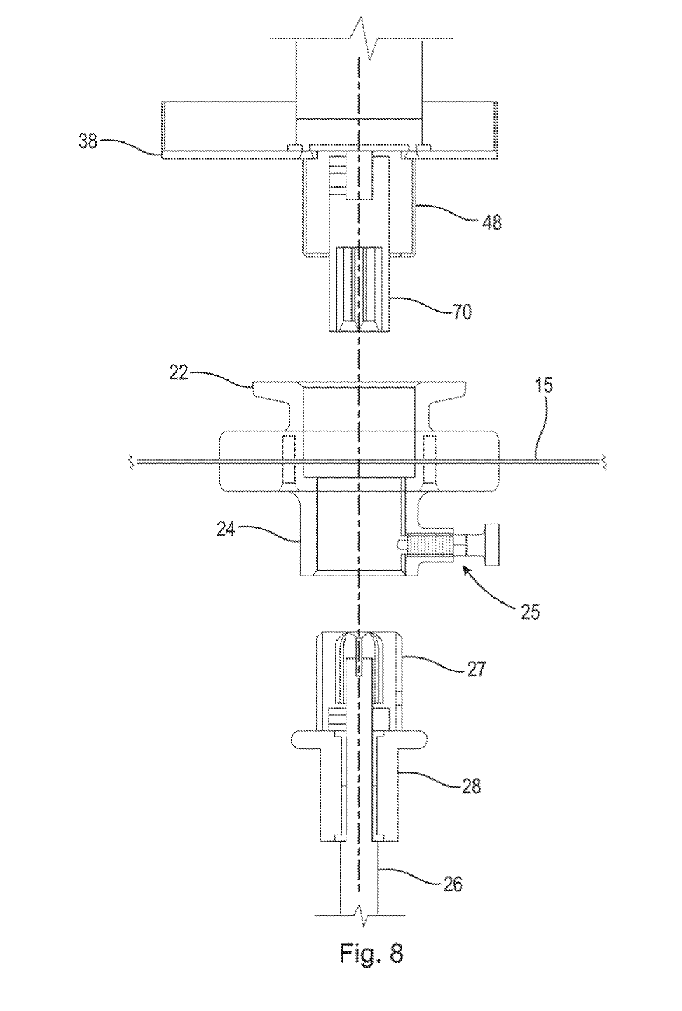

FIG. 8 is an exploded cross-section generally of area 8 in FIG. 4. The following should be view of FIGS. 2 through 8, and provides further detail regarding stirring assembly 100. Vessel 11 can be any vessel known in the art, that is, vessel 11 is not limited to a particular size, configuration, or material of construction. The following discussion presents a non-limiting example of a possible configuration of assembly 100. Although a specific configuration is presented, it should be understood that assembly 100 is not limited to the configuration shown. Handles 12a and 12b are for lifting the vessel. Two latch strikes 18a and 18b with, for example, ⅜″ vertical pins 14a and 14b welded to lip 66 of the vessel, for example, adjacent to the handles 12a and 12b, are used to secure the lid. As noted above, vertical baffle plate 113 is secured to wall 54 by any means known in the art, for example, the baffle plate is welded to the interior wall. As further described below, the baffle plate prevents formation of an undesirable force vortex in the contents of the vessel as apparatus 56 stirs the contents, and increase desirable mixing of content in the vessel.

Housing 36 of the control unit can be made of any material known in the art, for example, stainless steel, ABS plastic, or aluminum and encloses motor drive, or power source 65. Motor drive 65 can be any motor drive known in the art. In an example embodiment, drive 65 is an AC/DC switching power supply, for example, converting 120 VAC to 48 VDC. In an example embodiment, drive 65 is a PSPPS-200-48 manufactured by Wall Industries. Handle 39 secured to the top of the motor can be used to lift the housing from and place the housing on top center receptacle plate 22 and supports input power line 41. The motor drive controls operation of motor 58, which in turn is operably connected to rotation shaft 26, for example, via coupling 48. That is, motor 58 rotates the shaft.

Operation of shaft 26 is controlled by control unit 35. On/off switch 43 controls connection of the motor to the power supply. Variable speed switch 44 is used to control the speed at which the motor operates and the rotational speed of the shaft, that is, the mixing speed of apparatus 56. Portion 38, in one example, made of aluminum, of housing 36 includes snap lock connector 37. Top center receptacle 22 provides a plate support for housing 36, and acts as a handle for placing the lid assembly on the vessel or lifting the lid assembly off the vessel. Receptacle 22 also receives motor coupling 48 and motor housing snap lock connector 37 and is secured to lid 15 by manual spring plunger 23. Manual spring plunger 23 is operable by a user, typically without the use of tools, to release snap lock connector 37.

Bottom center receptacle 24 receives the stirrer/mixer snap lock connector 28 and is secured by manual spring plunger 25, for example, end 27 of connector 28 interfaces with portion 70 of coupling 48. Manual spring plunger 25 is operable by a user, typically without the use of tools, to release snap lock connector 28. To deposit and secure lid assembly 57, to the vessel, the lid is positioned so holes 21a and 21b are aligned with vertical pins 14a and 14b and lowered to contact lip 66 of the vessel. The lever cam latches 18a and 18b are turned up to 90 degrees to secure the lid assembly to vessel strikes 19a and 19b.

The lid assembly includes opening 16 for introducing food ingredients, visual inspection and tasting of food product through out the cooking process, and supporting secondary lid 49 including handle 51. An insert 29, for example, made of plastic, is attached to distal end 60 of the shaft and rotates on bottom 61 of the vessel. Scraper blade 32, in one case, made of plastic, is press fit on shaft 34. The opposite end shaft 34 is press fit into the bearing support bracket 67. Bearing support bracket 67 is fixed to, for example, welded to, rotation shaft 26. A square pitch axial flow propeller 62 is secured to rotation shaft 26. In an example, embodiment, a position of the propeller along the shaft is adjustable, for example, the propeller is slideable along the shaft and a position of the propeller can be fixed by use of any means known in the art, for example, setscrews passing through the propeller and engaging the shaft.

It should be understood that various combinations of components of assembly 100 are possible. For example, apparatus 56 could be configured for use with a vessel other than vessel 11. In addition, vessel 11 with baffle plate 113 could be used with other than apparatus 56. In an example embodiment, assembly 100 is a slow cooker, for example, what is commonly referred to as a “crock pot.” Assembly 56 controls the functions of chopping, stirring and pureeing associated with propeller 62 and blade 32. Unit 35 can include: temperature control for a heating unit (not shown) for the assembly, including temperature control based on input from sensor 52; a clock; a timer for setting cooking and stirring times; and one or more alarms, for example, regarding an over-temperature condition.

In an example embodiment, motor 58 runs on a re-chargeable battery (not shown). In an example embodiment, blade 32 is a cutting blade for chopping and/or pureeing contents of the vessel.

Although stirring assembly 100 has been described with respect to cooked food product, it should be understood that assembly 100 also can be used in non-cooking applications. For example, assembly 100 can be used to blend cold emulsions such as salad dressings, sauces, vinaigrettes, marinades, mayonnaise, batters, and food gum hydration.

As noted above, in many commercial cooking operations it is necessary to provide continuous stirring and mixing of food product to avoid burning and or scorching during the cooking cycle. As a result of distractions that often occur in an active kitchen environment, such continuous stirring and mixing of food product is difficult to achieve by hand. Advantageously, assembly 100, by continuous mechanical stirring and mixing of food product in a closed vessel while controlling speed, time and temperature optimizes time management, reduces cook time, increases yields, improves consistency of food product, and eliminates error. Assembly 100 has at least the following advantages and benefits:
1. Stir, mix, blend and agitate low and high viscosity (hot and cold) liquids.
2. Low shear mixing—maintaining food product particulate integrity.
3. Control food product stirring speed (for example, 0 to 415 RPM).
4. Control cooking time and temperature.
5. Improve finished food product consistency.
6. Higher yield. Less evaporation. For example, complete a soup without removing the lid.
7. Reduce cooking mistakes.
8. Reduce labor. Allow cooks to focus on other important tasks.
9. Fast cook up. Generates high level of heat transfer.
10. Reduce food lost from burning or scorching
11. Blends cold emulsion.
12. Usable in a wide variety of application from domestic kitchens to commercial restaurant kitchens and Research and Development kitchens.

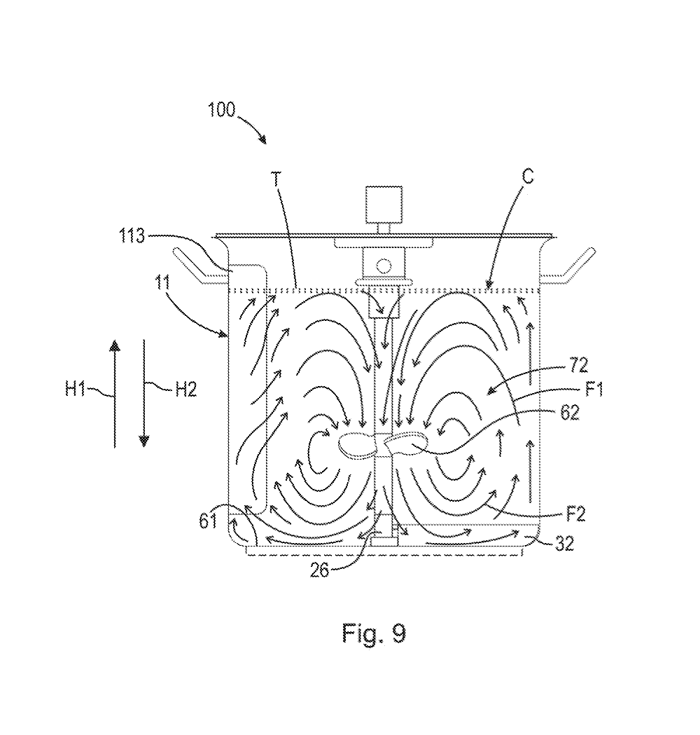

FIG. 9 is a side view of mixing patterns generated by the cooking vessel and stirring apparatus. As noted above, known stirring devices fail to adequately, mix and stir contents of a cooking vessel. For example, stratified layers, in the form of a vortex are formed and particulate in the contents of the vessel is not displaced horizontally through the content. Specifically, the heaviest particulate at the bottom of the vessel remains at the bottom of the vessel. Advantageously, the configuration of assembly 100 creates flow pattern 72 from bottom surface 61 to top layer T of contents C. The location of the propeller between the top and bottom of the vessel, in combination with the pitch of the propeller create flow in directions H1 and H2, rather than the stratified layers shown in FIG. 1.

Specifically, it is well known that a square pitch axial flow propeller operates by drawing the medium in which the propeller is immersed to the propeller (flow line F1) and then displacing the drawn medium past the propeller (flow line F2). This action creates the “circular” flow pattern shown by F1 and F2. The asymmetrical configuration of scraper 32 (a single scraper extends from the shaft) further acts to prevent stratified layers such as those shown in FIG. 1, while scrapping particulate from the bottom surface. The single scraper also adds an asymmetrical turbulence to the flow pattern created by the propeller, further enhancing mixing of the contents. Baffle plate 113 further accentuates the desirable non-stratified turbulence created by the propeller and the scraper. That is, the flow pattern created by the propeller is desirably disturbed, causing further desirable mixing. For example, portions of the flow pattern are diverted by contact with the baffle, also altering the flow pattern. Thus, contents C are thoroughly mixed from top to bottom, while particulate is simultaneously scraped from bottom surface 61.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What is claimed is:

1. A stirring assembly, comprising:
a vessel, including a baffle plate fixed to an interior wall of the vessel; and,
a stirring apparatus, including:
a lid assembly including a motor, the lid assembly removeably securable to the vessel;
a shaft connected to the motor and extending into an interior space of the vessel; and,
a single scrapper attached to a distal end of the shaft, extending radially outward from the shaft, and in contact with a bottom interior surface of the vessel.

2. The stirring assembly of claim 1, wherein:
the stirring apparatus includes a multi-bladed propeller secured to the shaft; and,
a position of the propeller along a length of the shaft is adjustable.

3. The stirring assembly of claim 1, wherein a side of the baffle plate facing the interior wall is in continuous contact with the interior wall.

4. The stirring assembly of claim 1, wherein the baffle plate is orthogonal to the interior wall.

5. The stirring assembly of claim 1, wherein:
the motor is a variable speed motor; and,
the lid assembly includes:
a thermal sensor extending into the interior space of the vessel; and,
a control unit with:
a variable speed control unit for controlling a speed of the motor; and,
a temperature display for displaying output from the thermal sensor.

6. The stirring assembly of claim 5, wherein the variable speed motor operates between zero revolutions per minute (rpm) and 415 rpm.

7. The stirring apparatus of claim 1, wherein the propeller and the shaft rotate in unison.

8. A cooking vessel, comprising:
an interior wall; and,
a baffle plate fixedly connected to the interior wall.

9. The cooking vessel of claim 8, wherein a side of the baffle plate facing the interior wall is in continuous contact with the interior wall.

10. The cooking vessel of claim 8, wherein the baffle plate is substantially orthogonal to the interior wall.

11. The cooking vessel of claim 8, further comprising:
a stirring apparatus, including:
a lid assembly including a variable speed motor, the lid assembly removeably secureable to the vessel;
a shaft removeably connected to the motor and extending into an interior space of the vessel;
a single scrapper attached to a distal end of the shaft, extending radially outward from the shaft, and in contact with a bottom interior surface of the vessel; and,
a multi-bladed propeller secured to the shaft, wherein a position of the propeller along a length of the shaft is adjustable.

12. The cooking vessel of claim 11, wherein the stirring apparatus includes:
a thermal sensor extending into an interior space of the vessel; and,
a control unit with:
a variable speed control unit for controlling a speed of the motor; and,
a temperature display for displaying output from the thermal sensor.

13. The cooking vessel of claim 11, wherein the motor operates between zero revolutions per minute (rpm) and 415 rpm.

14. The cooking vessel of claim 11, wherein the propeller is fixed to the shaft so that the propeller and the shaft rotate in unison.

15. A method for stirring contents of a cooking vessel, comprising:
positioning a propeller along a length of a shaft;
extending the shaft into an interior space of the cooking vessel;
contacting a bottom surface of the interior space with a single scrapper, attached to a distal end of the shaft and extending radially outward from the shaft; and,
rotating, using a motor, the propeller and the scraper such that:
a first portion of the contents located between the propeller and a top of the vessel, in a direction parallel to the shaft, is drawn toward the propeller and displaced past the propeller toward the bottom surface; and,
a second portion of the contents in contact with the scraper is displaced upward and beyond the propeller in the direction parallel to the shaft.

16. The method of claim 15, wherein the motor is a variable speed motor, the method further comprising:
measuring a temperature of the contents;
displaying the temperature on a control unit; and,
varying a speed of the motor using the control unit.

17. The method of claim 15, wherein varying the speed of the motor using the control unit includes operating the motor between zero revolutions per minute (rpm) and 415 rpm.

18. The method of claim 15, wherein positioning a propeller along a length of a shaft includes adjustably positioning the propeller along the length of the shaft.
